(19) United States
(12) Patent Application Publication
(10) Pub. No.: US 2010/0239445 A1
(43) Pub. Date: Sep. 23, 2010

(54) FLANGE OF A HIGH-PRESSURE FUEL PUMP

(76) Inventor: Friedrich Boecking, Stuttgart (DE)

Correspondence Address: RONALD E. GREIGG;GREIGG & GREIGG P.L.L.C., 1423 POWHATAN STREET, UNIT ONE, ALEXANDRIA, VA, 22314, US

(21) Appl. No.: 12/682,417

(22) PCT Filed: Sep. 18, 2008
(86) PCT No.: PCT/EP2008/062415

§ 371 Date: Apr. 9, 2010

(30) Foreign Application Priority Data
Oct. 11, 2007 (DE) 102007048853.1

(51) Int. Cl. A61M 1/00

(52) U.S. Cl. 417/437

(57) ABSTRACT

The invention relates to a high-pressure fuel pump for operating an internal combustion engine, with the pump being connected to a low-pressure and a high-pressure circuit. The pump includes a pump housing and a pump flange with which the pump is detachably fastened to the internal combustion engine. A pump camshaft is provided in order to compress fuel from the low-pressure circuit by way of the movement of at least one pump piston and to deliver the compressed fuel to the high-pressure circuit. The pump flange is further divided into an inner and an outer part, in a manner such that the inner part receives a bearing of the pump camshaft and the outer part receives elements of the low-pressure circuit.

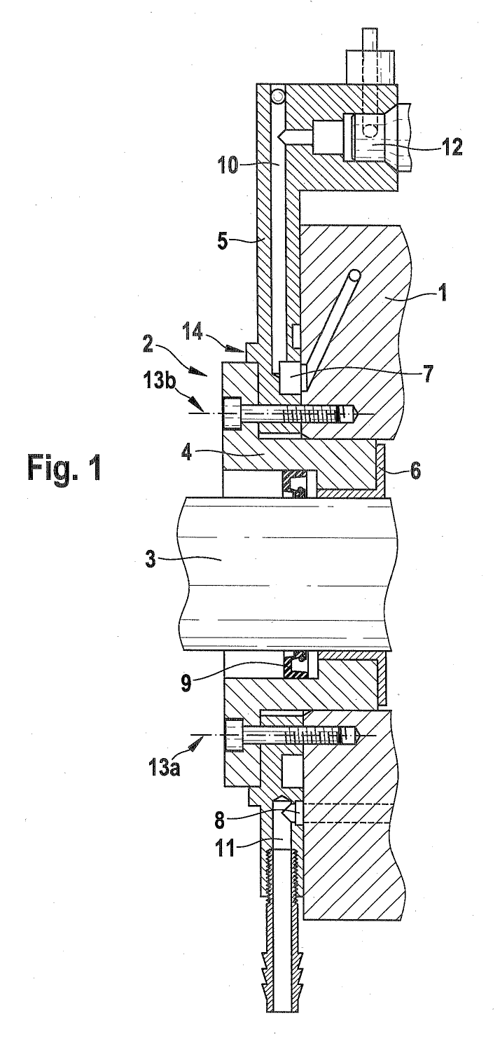

The invention relates to a high-pressure fuel pump for operating an internal combustion engine, communicating with a low-pressure circuit and a high-pressure circuit and comprising a pump housing and a pump flange, by way of which flange the pump is secured detachably to the engine, and a pump camshaft is provided for compressing fuel from the low-pressure circuit, by means of the motion of at least one pump piston, and delivering it to the high-pressure circuit.

### PRIOR ART

From International Patent Disclosure WO 2005/052356 A2, a high-pressure fuel pump is already known, which comprises a housing, in which a pump piston is disposed, and a flange, disposed on the face end of the pump housing. A pump drive shaft is furthermore supported in the flange and housing and imposes a motion on the pump piston via an eccentric and a polygonal embodiment. A pressure chamber located above the pump piston communicates on the one hand with an inlet of a low-pressure circuit and on the other with a high-pressure valve of a high-pressure circuit.

In the embodiment of a high-pressure fuel pump disclosed in the prior art, the flange on the one hand supports a bearing point of the pump drive shaft and on the other has lines of the inlet of the low-pressure circuit to the pressure chamber of the pump piston.

In such an embodiment, the effort and expense for producing such a flange are disadvantageous.

It is therefore the object of the present invention to create a flange of a high-pressure fuel pump, which flange makes a bearing point and a low-pressure inlet available and in which the production cost is reduced.

According to the invention, this object is attained in that the pump flange is subdivided into an inner part and an outer part, and the inner part receives a bearing of the pump camshaft, and the outer part receives elements of the low-pressure circuit. By this provision, the inner part of the pump flange can be produced as a rotationally symmetrical component, while the outer part can be adapted to the given aspects and connections of the pump housing and of the engine. As a result of the rotationally symmetrical embodiment of the inner part, greater precision in manufacture can be attained that suits the demands for guidance of the pump camshaft.

In a refinement of the invention, the outer part of the pump flange has annular grooves for communication with fuel lines of the pump housing. It is advantageous in this respect that the angular position of the outer part of the pump flange relative to the pump housing can be selected freely. Hence the pump flange can be put into a position that is optimal for the space available.

In a further feature of the invention, the inner part of the pump flange has at least one wave sealing ring. It is advantageous in this respect that the disposition of one or more wave sealing rings in this region effects optimal sealing off of the pump from the environment.

In a refinement of the invention, an inlet, a return and a metering unit of the low-pressure circuit are integrated with the outer part of the pump flange. The integration of these elements makes it possible to economize on material for lines and the like outside the flange.

In a further feature of the invention, the inner and outer parts of the pump flange are detachably secured to the pump housing via matching fastening points. By this provision, the number of fastening points is reduced, and furthermore, assembly is made easier.

In a refinement of the invention, the outer part is guided via the inner part of the pump flange. This has the advantage that the effort and expense of mounting of the two parts of the pump flange on the pump housing is reduced substantially.

In a further feature of the invention, the outer part has a rib for guidance on a face end oriented toward the inner part. It is advantageous here that this kind of guidance can be attained at low production cost.

In a refinement of the invention, the outer part of the pump flange has screw-on points, on the side toward the engine. As a result, a good capability of securing the high-pressure fuel pump to the engine is created.

Further provisions that improve the invention are described in further detail below together with the description of a preferred exemplary embodiment of the invention in conjunction with a drawing.

### EXEMPLARY EMBODIMENT

The sole FIGURE of the drawing shows a sectional view of the flange region of the high-pressure fuel pump, taken along the pump camshaft.

The pump flange 2, comprising the inner part 4 and the outer part 5, is disposed on the face end on the pump housing 1; some regions of the inner part of the pump flange 4 protrude into the pump housing 1 in order to receive the bearing 6 of the pump camshaft 3. In a recess in the inner part 4 of the pump flange 2 in front of the bearing 6, a wave sealing ring 9 is also disposed. The outer part 5 is placed on the outer contour of the inner part 4 and axially between the inner part 4 and the pump housing 1, and the outer part is guided on the inner part 4 via the rib 14. The inner part 4 and the outer part 5 are connected to the pump housing 1 via the fastening points 13a and 13b.

A metering unit 12, which regulates the fuel quantity of the high-pressure pump, is integrated with the outer part 5. Beginning at this metering unit 12, an inlet 10 leads to an annular groove 7, which is in coincidence with a fuel line of the pump housing 1. The fuel line of the pump housing 1 is also in communication with one or more pressure chambers of the high-pressure fuel pump, in which chambers the fuel at high pressure is generated when the pump is in operation. A return line of the pump housing 1 also communicates with an annular groove 8 and with the return 11 as well, to enable returning leakage fuel to the fuel tank, not shown.

1-8. (canceled)

9. A high-pressure fuel pump for operating an internal combustion engine, communicating with a low-pressure circuit and a high-pressure circuit and comprising:
a pump housing and a pump flange, by way of which flange the pump is secured detachably to the engine; and
a pump camshaft for compressing fuel from the low-pressure circuit, by means of the motion of at least one pump piston, and delivering the fuel to the high-pressure circuit, the pump flange being subdivided into an inner part and an outer part, wherein the inner part receives a bearing of the pump camshaft, and the outer part receives elements of the low-pressure circuit.

10. The high-pressure fuel pump as defined by claim 9, wherein the outer part of the pump flange has annular grooves for communication with fuel lines of the pump housing.

11. The high-pressure fuel pump as defined by claim 9, wherein the inner part has at least one wave sealing ring.

12. The high-pressure fuel pump as defined by claim 10, wherein the inner part has at least one wave sealing ring.

13. The high-pressure fuel pump as defined by claim 9, wherein an inlet, a return, and a metering unit of the low-pressure circuit are integrated with the outer part.

14. The high-pressure fuel pump as defined by claim 10, wherein an inlet, a return, and a metering unit of the low-pressure circuit are integrated with the outer part.

15. The high-pressure fuel pump as defined by claim 11, wherein an inlet, a return, and a metering unit of the low-pressure circuit are integrated with the outer part.

16. The high-pressure fuel pump as defined by claim 12, wherein an inlet, a return, and a metering unit of the low-pressure circuit are integrated with the outer part.

17. The high-pressure fuel pump as defined by claim 9, wherein the inner part and the outer part are secured detachably to the pump housing via matching fastening points.

18. The high-pressure fuel pump as defined by claim 11, wherein the inner part and the outer part are secured detachably to the pump housing via matching fastening points.

19. The high-pressure fuel pump as defined by claim 16, wherein the inner part and the outer part are secured detachably to the pump housing via matching fastening points.

20. The high-pressure fuel pump as defined by claim 9, wherein the outer part is guided via the inner part.

21. The high-pressure fuel pump as defined by claim 11, wherein the outer part is guided via the inner part.

22. The high-pressure fuel pump as defined by claim 19, wherein the outer part is guided via the inner part.

23. The high-pressure fuel pump as defined by claim 20, wherein the outer part, for guidance on a face end oriented toward the inner part, has a rib.

24. The high-pressure fuel pump as defined by claim 21, wherein the outer part, for guidance on a face end oriented toward the inner part, has a rib.

25. The high-pressure fuel pump as defined by claim 22, wherein the outer part, for guidance on a face end oriented toward the inner part, has a rib.

26. The high-pressure fuel pump as defined by claim 9, wherein the outer part has screw-on points, on the side toward the engine, to attach elements of the low-pressure circuit.

27. The high-pressure fuel pump as defined by claim 11, wherein the outer part has screw-on points, on the side toward the engine, to attach elements of the low-pressure circuit.

28. The high-pressure fuel pump as defined by claim 25, wherein the outer part has screw-on points, on the side toward the engine, to attach elements of the low-pressure circuit.
